(19) United States
(12) Patent Application Publication
(10) Pub. No.: US 2019/0333633 A1
(43) Pub. Date: Oct. 31, 2019

(54) MEDICAL DEVICE INFORMATION PROVIDING SYSTEM, MEDICAL DEVICE INFORMATION PROVIDING METHOD, AND PROGRAM

(71) Applicant: OPTiM Corporation, Saga-shi (JP)

(72) Inventor: Shunji SUGAYA, Tokyo (JP)

(21) Appl. No.: 16/309,549

(22) PCT Filed: Jun. 16, 2016
(86) PCT No.: PCT/JP2016/067966

§ 371 Date: Dec. 13, 2018

(51) Int. Cl. G16H 40/40; G06K 9/00

(52) U.S. Cl. CPC G16H 40/40; G06K 7/1417; G06K 9/00671

(57) ABSTRACT

An object of the present invention is to provide a medical device information providing system, a medical device information providing method, and a program capable of easily understanding a necessary measure at the time of malfunction. A medical device information providing system 1 for accessing a storage unit that stores medical device information to acquire and display the medical device information specifies an ID of a medical device appearing in a captured image captured by a terminal 100 or a medical device associated with a two-dimensional code read by the terminal 100, acquires medical device information associated with the specified ID of the medical device from the storage unit, and displays the acquired medical device information on a display unit.

### TECHNICAL FIELD

The present invention relates to a medical device information providing system, a medical device information providing method, and a program for accessing a storage unit storing medical device information to acquire and display the medical device information.

### BACKGROUND ART

Conventionally, when a malfunction or failure occurs in a medical device installed in a medical field, a medical staff is needed to see a sticker, a manual or the like attached to the medical device, call to the described phone number of a maintenance manager, and request a necessary action.

In addition, although the malfunction does not occur, a configuration of acquiring and viewing an instruction or safety confirmation information on a medical device from a server by reading, by a terminal, a two-dimensional code attached to the medical device is disclosed (see Patent Document 1).

### PRIOR ART DOCUMENT

### Patent Document

Patent Document 1: Japanese Patent Application Publication No. 2013-200752

### SUMMARY OF THE INVENTION

### Technical Problem

However, in the configuration of Patent Document 1, it is possible to view information on use of the medical device such as a name, an instruction, and the like. However, since the medical staff cannot know the necessary measure at the time of malfunction of the medical device, it is difficult to easily understand the necessary measure.

It is an object of the present invention to provide a medical device information providing system, a medical device information providing method, and a program capable of easily understanding the necessary measure at the time of malfunction.

### Technical Solution

The present invention provides the following solutions.

An invention according to a first aspect provides a medical device information providing system for accessing a storage unit that stores medical device information to acquire and display the medical device information, the medical device information providing system including a medical device specifying unit that specifies an ID of a medical device appearing in a captured image captured by a terminal by image recognition, a medical device information acquiring unit that acquires medical device information associated with the specified ID of the medical device from the storage unit, and a display unit that displays the acquired medical device information on a display unit.

According to the first aspect of the invention, a medical device information providing system for accessing a storage unit that stores medical device information to acquire and display the medical device information specifies an ID of a medical device appearing in a captured image captured by a terminal by image recognition, acquires medical device information associated with the specified ID of the medical device from the storage unit, and displays the acquired medical device information on a display unit.

Here, the invention according to the first aspect is a category of a medical device information providing system, but even in other categories such as a method or a program, exhibits the same action and effect corresponding to the category.

An invention according to a second aspect provides a medical device information providing system for accessing a storage unit that stores medical device information to acquire and display the medical device information, the medical device information providing system including a medical device specifying unit that specifies an ID of a medical device associated with a two-dimensional code read by a terminal, a medical device information acquiring unit that acquires medical device information associated with the specified ID of the medical device from the storage unit, and a display unit that displays the acquired medical device information on a display unit.

According to the second aspect of the invention, a medical device information providing system for accessing a storage unit that stores medical device information to acquire and display the medical device information specifies an ID of a medical device associated with a two-dimensional code read by a terminal, acquires medical device information associated with the specified ID of the medical device from the storage unit, and displays the acquired medical device information on a display unit.

Here, the invention according to the second aspect is a category of a medical device information providing system, but even in other categories such as a method or a program, exhibits the same action and effect corresponding to the category.

An invention according to a third aspect provides a medical device information providing system which is the invention according to the first or second aspect, wherein the display unit displays a change in the acquired medical device information as a graph.

According to the third aspect of the invention, a medical device information providing system, which is the invention according to the first or second aspect, displays a change in the acquired medical device information as a graph.

An invention according to a fourth aspect provides a medical device information providing system which is the invention according to the first aspect, wherein the display unit displays the acquired medical device information in association with position information of a location where the terminal captures the captured image.

According to the fourth aspect of the invention, a medical device information providing system, which is the invention according to the first aspect, displays the acquired medical device information in association with position information of a location where the terminal captures the captured image.

An invention according to a fifth aspect provides a medical device information providing system which is the invention according to the second aspect, wherein the display unit displays the acquired medical device information in association with position information of a location where the terminal reads the two-dimensional code.

According to the fifth aspect of the invention, a medical device information providing system, which is the invention according to the second aspect, displays the acquired medical device information in association with position information of a location where the terminal reads the two-dimensional code.

An invention according to a sixth aspect provides a medical device information providing system, which is the invention according to the first aspect, further including an evidence utilization unit that uses the captured image as an evidence of the specified medical device when accessing the storage unit.

According to the sixth aspect of the invention, a medical device information providing system, which is the invention according to the first aspect, uses the captured image as an evidence of the specified medical device when accessing the storage unit.

An invention according to a seventh aspect provides a medical device information providing system, which is the invention according to the second aspect, further including an evidence utilization unit that uses the two-dimensional code as an evidence of the specified medical device when accessing the storage unit.

According to the seventh aspect of the invention, a medical device information providing system, which is the invention according to the second aspect, uses the two-dimensional code as an evidence of the specified medical device when accessing the storage unit.

An invention according to an eighth aspect provides a medical device information providing system, which is the invention according to the first or second aspect, further including a calling unit that calls to a phone number included in the acquired medical device information.

According to the eighth aspect of the invention, a medical device information providing system, which is the invention according to the first or second aspect, calls to a phone number included in the acquired medical device information.

An invention according to a ninth aspect provides a medical device information providing method for accessing a storage unit that stores medical device information to acquire and display the medical device information, the medical device information providing method including specifying an ID of a medical device appearing in a captured image captured by a terminal by image recognition, acquiring medical device information associated with the specified ID of the medical device from the storage unit, and displaying the acquired medical device information on a display unit.

An invention according to a tenth aspect provides a medical device information providing method for accessing a storage unit that stores medical device information to acquire and display the medical device information, the medical device information providing method including specifying an ID of a medical device associated with a two-dimensional code read by a terminal, acquiring medical device information associated with the specified ID of the medical device from the storage unit, and displaying the acquired medical device information on a display unit.

An invention according to an eleventh aspect provides a program for causing a medical device information providing system for accessing a storage unit that stores medical device information to acquire and display the medical device information to execute specifying an ID of a medical device appearing in a captured image captured by a terminal by image recognition, acquiring medical device information associated with the specified ID of the medical device from the storage unit, and displaying the acquired medical device information on a display unit.

An invention according to a twelfth aspect provides a program for causing a medical device information providing system for accessing a storage unit that stores medical device information to acquire and display the medical device information to execute specifying an ID of a medical device associated with a two-dimensional code read by a terminal, acquiring medical device information associated with the specified ID of the medical device from the storage unit, and displaying the acquired medical device information on a display unit.

### Effects of the Invention

According to the present invention, it is possible to provide a medical device information providing system, a medical device information providing method, and a program capable of easily understanding a necessary measure at the time of malfunction.

### BRIEF DESCRIPTION OF THE DRAWINGS

FIG. 1 is a diagram showing an overview of a medical device information providing system 1.

FIG. 2 is an overall configuration diagram of a medical device information providing system 1.

FIG. 3 is a functional block diagram of a server 10 and an information terminal 100.

FIG. 4 is a diagram showing a medical device ID database creating process executed by a server 10 and an information terminal 100.

FIG. 5 is a diagram showing an image specifying process executed by a server 10 and an information terminal 100.

FIG. 6 is a diagram showing an image specifying process executed by a server 10 and an information terminal 100.

FIG. 7 is a diagram showing a code reading process executed by a server 10 and an information terminal 100.

FIG. 8 is a diagram showing a code reading process executed by a server 10 and an information terminal 100.

FIG. 9 is a diagram showing an example of medical device information displayed by an information terminal 100.

FIG. 10 is a diagram showing an example of an error notification displayed by an information terminal 100.

### DETAILED DESCRIPTION OF THE EMBODIMENTS

Hereinafter, embodiments for carrying out the present invention are described with reference to the drawings. It is to be understood that the embodiments are merely examples and the scope of the present invention is not limited to the disclosed embodiments.

Overview of Medical Device Information Providing System 1

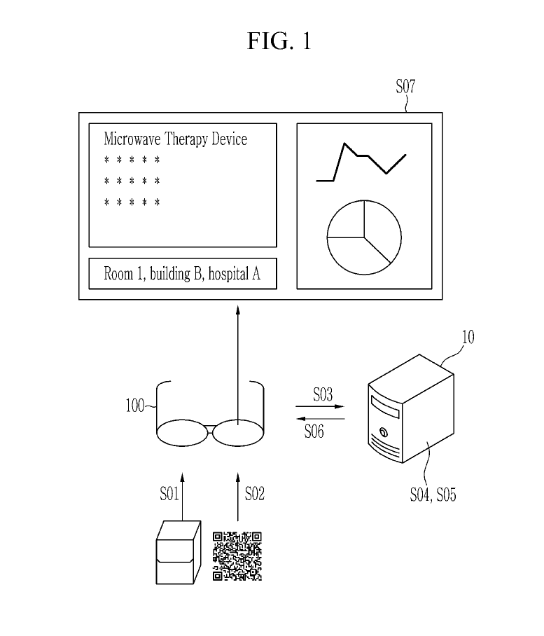

An overview of a preferred embodiment of the present invention is described with reference to FIG. 1. FIG. 1 is a diagram for explaining an overview of a medical device information providing system 1 which is a preferred embodiment of the present invention. The medical device information providing system 1 includes a server 10 and an information terminal 100.

The server 10 is a server device capable of performing data communication with the information terminal 100. The server 10 stores medical device information which is various information on the medical device in a storage unit of the server 10, accesses the storage unit to acquire the medical device information, and displays the medical device information on the information terminal 100.

The information terminal 100 is a terminal device operated by a medical staff and is capable of performing data communication with the server 10. The information terminal 100 is, for example, a mobile phone, a portable information terminal, a tablet terminal, a personal computer, an electric appliance such as a netbook terminal, a slate terminal, an electronic book terminal, or a portable music player, a wearable terminal such as a smart glasses or a head mount display, or other goods. In the following description, it is assumed that the information terminal 100 is the wearable terminal.

The information terminal 100 captures a captured image of a target medical device (step S01). In addition, the information terminal 100 reads an information code such as a two-dimensional code or a one-dimensional code of the target medical device (step S02). The processing in either step S01 or step S02 or in both step S01 and step S02 may be executed. The information terminal 100 executes capturing the medical device or reading the information code by an imaging capturing device such as a camera owned by the information terminal 100. The information terminal 100 captures a part or the whole of the medical device. The information terminal 100 reads the information code attached to the medical device. The information terminal 100 captures a captured image such as a moving image or a still image of the medical device. For example, the information terminal 100 captures the medical device as the moving image and obtains the captured image in which the captured moving image is converted to a plurality of still images. The processing in step S01 and step S02 may be accepted and executed by a voice or gesture of the medical staff, or may be executed by visually recognizing, by the medical staff wearing the information terminal 100, the target medical device or information code through the information terminal 100.

Further, the information terminal 100 may be configured to acquire its own position information and store the position information in its own storage unit at the time of executing the processing of step S01 or step S02. The information terminal 100 uses its own position information stored at this time in processing to be described below. Furthermore, the information terminal 100 may execute capturing the medical device or reading the information code by a configuration other than the image capturing device.

The information terminal 100 transmits captured image information indicating data of the captured image or code information indicating data of the information code to the server 10 (step S03).

The server 10 receives the captured image information or the code information and specifies an ID of the target medical device based on the received captured image information or code information (step S04). The server 10 performs image analysis on the captured image based on the captured image information, thereby extracting a feature amount of the medical device appearing in the captured image. The server 10 specifies the ID associated with the extracted feature amount of the medical device, based on the extracted feature amount of the medical device and a medical device ID database in which feature amounts of medical devices and IDs are associated with each other in advance. In addition, the server 10 reads information stored in the information code based on the code information and specifies an ID of the medical device associated with the stored information.

In a case where the ID of the medical device is stored in the code information, the server 10 may be configured to specify the ID of the medical device stored in the code information.

The server 10 acquires medical device information associated with the specified ID of the medical device (step S05). The server 10 extracts the medical device information associated with the specified ID of the medical device, based on the specified ID of the medical device and a medical device information database in which IDs of medical devices and medical device information are associated with each other in advance, and acquires the medical device information. The medical device information is, for example, various information on the medical device such as a purpose of use, effectiveness, an effect, an operation method, a usage method, a precaution on use, a use start date and time, a storage method, a holding method, a maintenance inspection item, a name of a manufacturer/distributor, a phone number of a manufacturer/distributor, and the like.

Further, the server 10 may be configured to use the captured image information or the code information as a specific evidence of the medical device. The server 10 may permit access to the medical device information database if the captured image or information code is genuine, and deny access to the medical device information database if it is not genuine.

The server 10 transmits the acquired medical device information to the information terminal 100 (step S06).

The information terminal 100 receives the medical device information and displays the medical device information on a display unit of the information terminal 100 (step S07). Further, the information terminal 100 may be configured to display a change in medical device information as a graph. Furthermore, the information terminal 100 may be configured to display the medical device information in association with the stored position information. In addition, the information terminal 100 may be configured to recognize a phone number included in the received medical device information as a text and make a telephone call.

The above is the overview of the medical device information providing system 1.

System Configuration of Medical Device Information Providing System 1

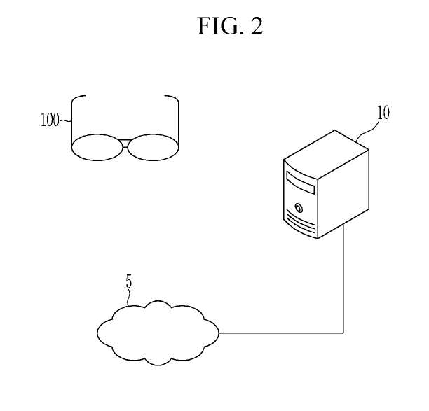

A system configuration of a medical device information providing system 1, which is a preferred embodiment of the present invention, is described with reference to FIG. 2. FIG. 2 is a diagram showing a system configuration of a medical device information providing system 1 which is a preferred embodiment of the present invention. The medical device information providing system 1 includes a server 10, an information terminal 100, a public line network (Internet network, third or fourth generation communication network, or the like) 5. In addition, the number of information terminal(s) 100 is not limited to one, and may be two or more. Further, the server 10 and the information terminal 100 are not limited to existing devices and may be virtual devices. Furthermore, each process to be described below may be realized by either the server 10 or the information terminal 100, or both the server 10 and the information terminal 100.

The server 10 is the above-described server device having functions to be described below.

The information terminal 100 is the above-described terminal device having functions to be described below. In the following description, it is assumed that the information terminal 100 is a wearable terminal.

Explanation of Functions

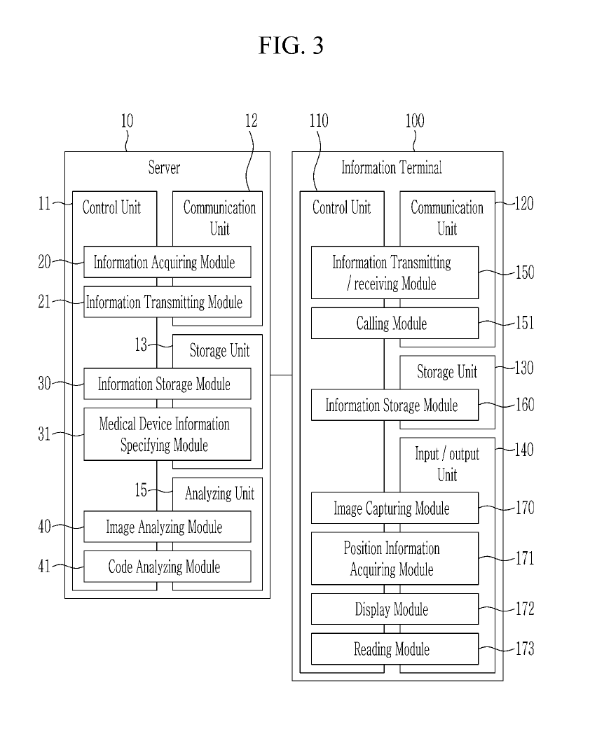

Functions of a medical device information providing system 1 which is a preferred embodiment of the present invention are described with reference to FIG. 3. FIG. 3 is a functional block diagram of a server 10 and an information terminal 100.

The server 10 includes, as a control unit 11, a CPU (Central Processing Unit), a RAM (Random Access Memory), a ROM (Read Only Memory), and the like, and includes, as the communication unit 12, a device for enabling communication with other devices, for example, a WiFi (Wireless Fidelity) compliant device conforming to IEEE 802.11. Further, the server 10 includes, as a storage unit 13, a data storage unit such as a hard disk, a semiconductor memory, a recording medium, a memory card, or the like. The storage unit 13 stores various databases to be described below. Furthermore, the server 10 includes, as an analyzing unit 15, an image analyzing device which executes image analysis of a captured image, an information code analyzing device which analyzes various types of information embedded in an information code, or the like.

In the server 10, the control unit 11 reads a predetermined program, thereby realizing an information acquiring module 20 and an information transmitting module 21 in cooperation with the communication unit 12. Further, in the server 10, the control unit 11 reads a predetermined program, thereby realizing an information storage module 30 and a medical device information specifying module 31 in cooperation with the storage unit 13. Furthermore, in the server 10, the control unit 11 reads a predetermined program, thereby realizing an image analyzing module 40 and a code analyzing module 41 in cooperation with the analyzing unit 15.

Like the server 10, the information terminal 100 includes, as a control unit 110, a CPU, a RAM, a ROM, and the like, includes, as a communication unit 120, a WiFi compatible device for enabling communication with other devices or a telephone device for calling a phone number, or the like, and includes, as a storage unit 130, a data storage unit. Further, the information terminal 100 includes, as an input/output unit 140, a display unit which outputs and displays data or images controlled by the control unit 110, an input unit, such as a touch panel, a keyboard, a mouse, a voice recognition device, a gesture recognition device, or the like, which accepts an input from a medical staff, an image capturing device which captures a captured image of the medical device, a code reading device which reads the information code, a position information acquiring device which acquires its own position information from a GPS or the like, or the like.

In the information terminal 100, the control unit 110 reads a predetermined program, thereby realizing an information transmitting/receiving module 150 and a calling module 151 in cooperation with the communication unit 120. Further, in the information terminal 100, the control unit 110 reads a predetermined program, realizing an information storage module 160 in cooperation with the storage unit 130. Furthermore, in the information terminal 100, the control unit 110 reads a predetermined program, thereby realizing an image capturing module 170, a position information acquiring module 171, a display module 172, and a reading module 173 in cooperation with the input/output unit 140.

Medical Device ID Database Creating Process

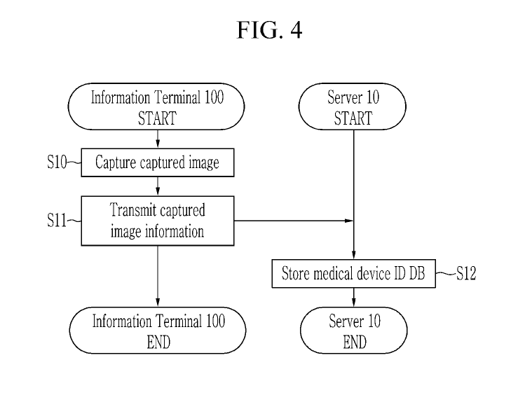

A medical device ID database creating process executed by a medical device information providing system 1 is described with reference to FIG. 4. FIG. 4 is a flowchart showing a medical device ID database creation process executed by a server 10 and an information terminal 100. The processing executed by modules of each device described above is described together with this processing.

An image capturing module 170 captures a captured image of a medical device (step S10). In step S10, the image capturing module 170 captures the captured image capable of identifying the medical device, such as a part or the whole of the medical device. The image capturing module 170 may configured to capture the medical device as a moving image and divide the captured moving image into a plurality of still images, or may be configured to capture a still image. The processing in step S10 is executed, for example, at the timing at which the medical device is delivered to the medical institution or at other timing. In a case of capturing the still image, it is preferable that the image capturing module 170 is configured to capture the medical device from a plurality of angles and capture a plurality of captured images. Further, in a case of capturing a moving image, it is preferable that the image capturing module 170 is configured to capture 360 degrees around the medical device and divide it into a plurality of still images.

An information transmitting/receiving module 150 transmits captured image information which is data of the captured image to the server 10 (step S11). In step S11, the information transmitting/receiving module 150 assigns an ID of the medical device to the captured image information and transmits the ID to the server 10. The ID of the medical device may be, for example, product information such as a product name, a manufacturing number, or the like, may be an identifier such as a character string inputted by a medical staff or a maintenance manager, may be a combination of the product information and the identifier, or may be other configurations.

An information acquiring module 20 acquires the captured image information and the ID. An information storage module 30 creates and stores a medical device ID database in which the captured image information and the ID is associated with each other (step S12).

The above is the medical device ID database creating process.

The above-described medical device ID database creating process may not have a configuration created by a medical staff, but may have a configuration in which the server 10 acquires and stores the above-described medical device ID database from an external server or the like. In this case, this process may be omitted and a process to be described below may be executed.

Image Specifying Process

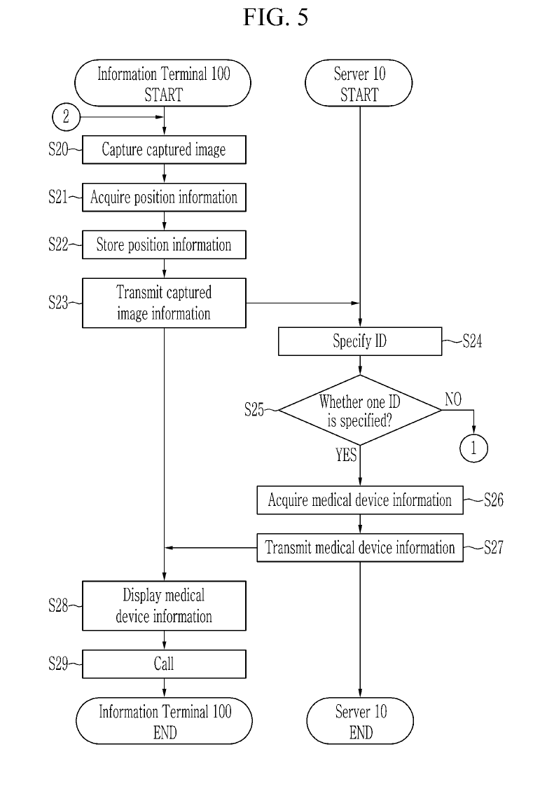

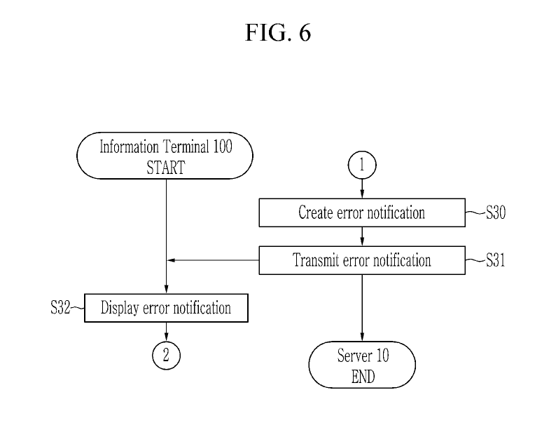

The image specifying process executed by a medical device information providing system 1 is described with reference to FIG. 5 and FIG. 6. FIG. 5 and FIG. 6 are flowcharts showing an image specifying process executed by a server 10 and an information terminal 100. The processing executed by modules of each device described above is described together with this processing.

An image capturing module 170 captures a captured image of a medical device (step S20). The processing in step S20 is the same as the processing in step S10 described above.

A position information acquiring module 171 acquires position information of an image capturing location (step S21). In step S21, the position information acquiring module 171 acquires the position information of the image capturing location by acquiring its own position information from a GPS or the like. Further, the position information acquiring module 171 may be configured to acquire its own position information by a sensor or the like, or may be configured to acquire its own position information by a configuration other than the GPS.

An information storage module 160 stores the acquired position information (step S22). The information storage module 160 may be configured to further store current date and time, information of a photographer, or other information in association with the position information, and may be configured to store only the position information.

Further, the information terminal 100 may be configured to execute the processing in step S21 and step S22 simultaneously with the processing in step S20. That is, when the image capturing module 170 captures the medical device, the position information acquiring module 171 may be configured to acquire the position information and the information storage module 160 may be configured to store the position information.

An information transmitting/receiving module 150 transmits captured image information which is data of the captured image to the server 10 (step S23). Further, the information transmitting/receiving module 150 may be configured to execute the processing in step S23 prior to the processing in step S22 described above.

An information acquiring module 20 acquires the captured image information. An image analyzing module 40 performs image analysis on the captured image based on the captured image information, thereby specifying an ID of the medical device appearing in the captured image (step S24). In step S24, the image analyzing module 40 extracts a feature amount of the medical device by the image analysis. The image analyzing module 40 performs the image analysis on a captured image based on captured image information stored in a medical device ID database and extracts a feature amount. The image analyzing module 40 compares the feature amount of the medical device extracted from the acquired captured image with the feature amount extracted from the stored captured image, and specifies the ID associated with the matched captured image information.

The image analyzing module 40 determines whether one ID has been specified (step S25). In step S25, the image analyzing module 40 determines whether one ID has been specified based on, for example, whether a plurality of medical devices appear in the captured image, whether there are a plurality of IDs associated with the matched feature amount, whether there is no ID associated with the matched feature amount, and the like.

In step S25, upon determining that one ID has been specified (YES in step S25), the image analyzing module 40 acquires medical device information associated with the specified medical device ID by accessing a medical device information database stored in the information storage module 30 (step S26). In step S26, the medical device information database is a database in which various information of the medical device is associated with the medical device ID. For example, the medical device information database may be configured based on medical device information and IDs inputted from the information terminal 100 by a medical staff, or may be configured based on medical device information and IDs inputted from another terminal device or the like, may be configured to be acquired from a database, a server, or the like possessed by a sales company, a maintenance manager, or the like of the medical device. The medical device information is the same as the above-described configuration.

In addition, in step S26, when accessing the medical device information database, a medical device information specifying module 31 may use the captured image information as a specific evidence. In this case, the medical device information specifying module 31 permits access to the above-described various databases and executes specifying the medical device information, when the medical device appears in the captured image. When the medical device does not appear in the captured image, the medical device information specifying module 31 denies access to the above-described various databases and does not execute specifying the medical device information.

An information transmitting module 21 transmits the acquired medical device information to the information terminal 100 (step S27).

The information transmitting/receiving module 150 receives the medical device information. A display module 172 displays the received medical device information (step S28). The display module 172 may be configured to display a part or the whole of the medical device information, may be configured to display a change in the medical device information as a graph, or may be configured to display the position information of the image capturing location stored in the information storage module 160 and the medical device information in association with each other. Further, the display module 172 may display the above-described configurations in combination, or may use other display manners. The part of the medical device information is, for example, information necessary for repairing the medical device, such as a name of the medical device, last maintenance date and time, a phone number of a sales company, a phone number of a maintenance manager, or the like, or other information. Further, the change in the medical device information is, for example, the number of times of use per day, the number of times of abnormality detection, or the like. Furthermore, the medical device information displayed by the display module 172 may be appropriately changed.

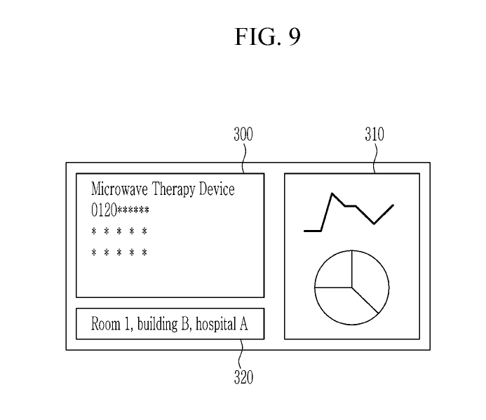

The medical device information displayed by the display module 172 is described with reference to FIG. 9. FIG. 9 is a diagram showing an example of the medical device information displayed by the display module 172. In FIG. 9, the display module 172 displays a medical device information display area 300, a graph display area 310, and a position information display area 320 on its display unit. The medical device information display area 300 is an area which displays the medical device information. An example of the medical device information displayed in the medical device information display area 300 is a name of a medical device, a name of a sales company, a phone number of a maintenance manager, a contact person in charge, or the like. The medical device information displayed in the medical device information display area 300 is a part or the whole of the received medical device information. In a case of displaying only the part, for example, a configuration in which only the medical device information which is designated in advance by the medical staff is displayed, a configuration in which necessary medical device information is determined by the information terminal 100 based on the current date and time, the current location, or the like and is displayed, or a configuration in which the medical device information to be displayed is designated based on an input operation such as a voice input or a gesture input and the designated medical device information is displayed may be used. The graph display area 310 is an area which displays a change in medical device information as a graph. The change in the medical device information displayed in the graph display area 310 may have, for example, a configuration in which a change in the number of times of use on a daily basis or a change in the number of times of abnormality detection on a daily basis is displayed as a bar graph, a configuration in which a change in a period during which maintenance and inspection is performed is displayed as a pie chart, or a configuration in which a frequency of abnormality detection is displayed as a line graph. The change in the medical device information displayed in the graph display area 310 may have a configuration in which the change in medical device information which is designated in advance by a medical staff is displayed or a configuration in which the medical device information to be displayed as the graph is designated based on an input operation such as a voice input or a gesture input and the change in the designated medical device information is displayed. Further, the position information display area 320 is an area which displays position information of an image capturing location. The position information displayed in the position information display area 320 is, for example, name, address, coordinates, identifier or the like of a location based on the position information. The position information display area 320 is an area which displays position information of a location where an information code is read in a code reading process to be described below.

On the other hand, all of the medical device information display area 300, the graph display area 310, and the position information display area 320 displayed by the display module 172 may not always need to be displayed, and any or a plurality of combinations of them may be displayed. Further, the medical device information to be displayed in the medical device information display area 300 can be appropriately changed. Furthermore, a type or content of the graph displayed in the graph display area 310 can be appropriately changed. In addition, a content of the position information displayed in the position information display area 320 can be appropriately changed. Additionally, the display module 172 may be configured to display information other than the above information. For example, the display module 172 may be configured to display a captured image or to display an information code to be described below, in addition to the medical device information.

Further, the display module 172 may be configured to initially display the medical device information display area 300, and then to receive an input operation such as a voice input or a gesture input from a medical staff and display the graph display area 310 or the position information display area 320. In other words, the display module 172 can freely select and display each of the above-described areas, and can also display a desired area at a desired timing.

A calling module 51 calls to a phone number included in the medical device information (step S29). In step S29, the calling module 51 text-recognizes a phone number related to a measure at the time of malfunction, such as a phone number of a distributor or a phone number of maintenance manager included in the received medical device information, activates an application or a device necessary for the call, and calls to this phone number. By doing in this way, the medical staff wearing the information terminal 100 can easily receive a support such as a request for repair or a necessary action at the time of malfunction.

On the other hand, in step S25, when determining that one ID has not been specified (NO in step S25), the image analyzing module 40 creates an error notification indicating this fact (step S30). In step S30, the error notification created by the image analyzing module 40 creates, for example, information indicating that a plurality of medical devices appear in the captured image, information indicating that a plurality of medical device IDs have been specified, information indicating a countermeasure for specifying one ID, the captured image information, information indicating a simple illustration on the countermeasure to be superimposed on the captured image, or the like. Further, the contents of the error notification created by the image analyzing module 40 can be appropriately changed.

The information transmitting module 21 transmits the created error notification to the information terminal 100 (step S31).

The information transmitting/receiving module 150 receives the notification. The display module 172 displays the error notification on its display unit (step S32).

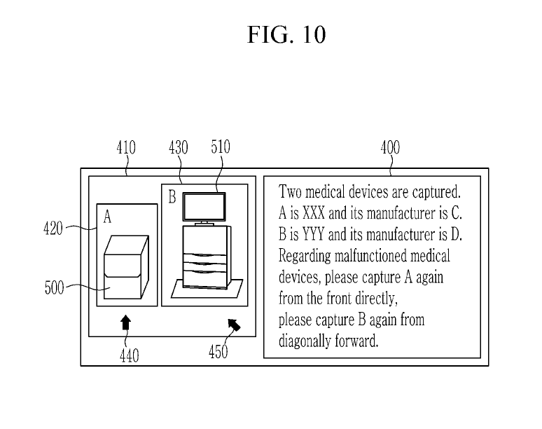

The error notification displayed by the display module 172 is described with reference to FIG. 10. FIG. 10 is a diagram showing an example of the error notification displayed by the display module 172. In FIG. 10, the display module 172 displays a notification content display area 400 and a captured image display area 410. The notification content display area 400 displays each information of the error notification created by the above-described image analyzing module 40 as a text. Specifically, in FIG. 10, the display module 172 displays a text indicating that a plurality of medical devices appear in the captured image, a text indicating that a plurality of medical device IDs are specified by the image analysis, and a text indicating an operation method necessary for specifying one medical device are displayed in the notification content display area 400. Further, the display module 172 displays a captured image in which the medical devices 500 and 510 appear, frame lines 420 and 430 indicating the specified medical devices, and countermeasure icons 440 and 450 that simply show countermeasures are displayed in the captured image display area 410. The display module 172 displays an identifier of the medical device within each of the frame lines 420 and 430. In FIG. 10, the display module 172 displays “A” and “B” as identifiers. In FIG. 10, the countermeasure icons 440 and 450 indicate that image capturing directions of the medical devices 500 and 510 are indicated.

On the other hand, all of the notification content display area 400 and the captured image display area 410 displayed by the display module 172 may not always need to be displayed, and any or both of them may be displayed. Further, the content displayed in the notification content display area 400 by the display module 172 is not limited to the text, and may be an icon, an illustration, or the like. Furthermore, the display manner of the error notification displayed by the display module 172 can be appropriately changed. In addition, positions and sizes of the notification content display area 400 and the captured image display area 410 displayed by the display module 172 can be appropriately changed. Additionally, the display module 172 may be configured to output this information by a voice based on the information of the error notification. Further, the display module 172 may be configured to display the error notification according to a display manner other than the text or voice. Furthermore, the display module 172 may be configured to display information on the error other than the above-described configurations.

The medical staff wearing the information terminal 100 captures the captured image of the medical device again based on the contents of the error notification. In other words, the information terminal 100 executes the above-described processing of step S20 again.

The above is the image specifying process.

In the above-described image specifying process, the processing executed by the server 10 or the information terminal 100 may be configured to be collaboratively executed by a the server 10 and the information terminal 100. Alternatively, a part or the whole of the processing executed by the server 10 may be configured to be executed by the information terminal 100, or a part or the whole of the processing executed by the information terminal 100 may be configured to be executed by the server 10.

Code Reading Process

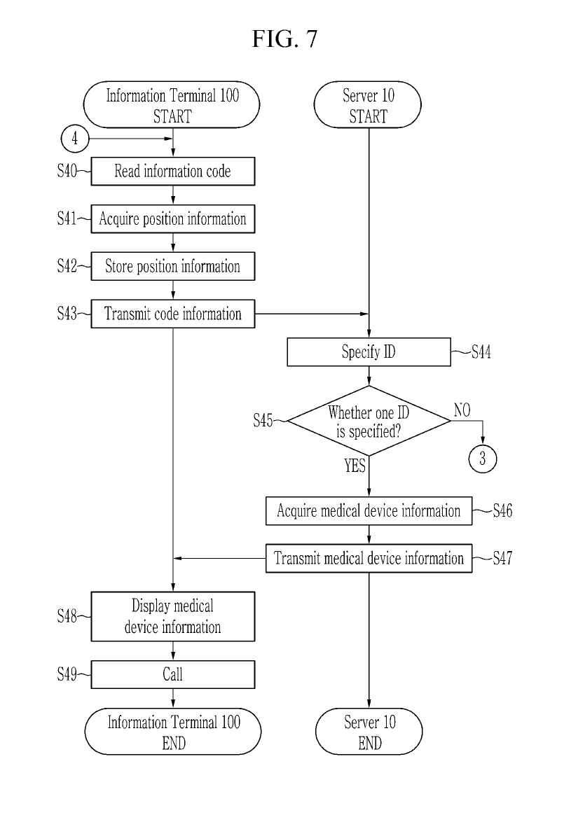

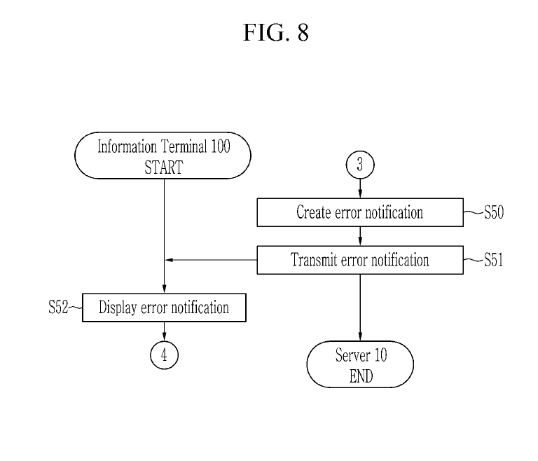

A code reading process executed by a medical device information providing system 1 is described with reference to FIG. 7 and FIG. 8. FIG. 7 and FIG. 8 are flowcharts showing a code reading process executed by a server 10 and an information terminal 100. The processing executed by modules of each device described above is described together with this processing. A detailed description of the same processing as the above-described image specifying process is omitted.

A reading module 173 reads an information code such as a two-dimensional code or a one-dimensional code of a medical device (step S40). In step S40, the reading module 173 reads the information code attached to a part of the medical device or the information code attached to a mounting table or the like on which the medical device is mounted, by a dedicated application, an image capturing device, or the like. In step S40, the information code read by the reading module 173 is not limited to the above-described configuration, and can be appropriately changed. Further, the information code may not need to be attached to the medical device itself, the mounting table, or the like, and can be appropriately changed.

A position information acquiring module 171 acquires position information of a reading location (step S41). The processing in step S41 is the same as the processing in step S21 described above.

An information storage module 160 stores the position information acquired by the position information acquiring module 171 in step S41 (step S42). The processing in step S42 is the same as the processing in step S22 described above.

Further, the information terminal 100 may be configured to execute the processing in step S41 and step S42 simultaneously with the processing in step S40. In other words, when the reading module 173 reads the information code, the position information acquiring module 171 may acquire the position information and the information storing module 160 may store the position information.

An information transmitting/receiving module 150 transmits the code information which is the data of the information code to the server 10 (step S43). Further, the information transmitting/receiving module 150 may be configured to execute the processing in step S43 prior to the processing in step S42 described above.

An information acquiring module 20 acquires the code information. Based on the code information, a code analyzing module 41 specifies an ID of the medical device associated with this code information (step S44). The code analyzing module 41 specifies the ID of the medical device by extracting the ID stored in the code information based on the code information.

The image analyzing module 40 determines whether one ID has been specified (step S45). In step S45, the image analyzing module 40 determines whether one ID has been specified based on, for example, whether a plurality of information codes have been read, whether there is no ID associated with the extracted ID, or the like.

In step S45, when determining that one ID has been specified (YES in step S45), the image analyzing module 40 acquires whether medical device information associated with the specified medical device ID by accessing a medical device information database stored in the information storage module 30 (step S46). The processing in step S46 is the same as the processing in step S26 described above.

In step S46, when accessing the medical device information database, a medical device information specifying module 31 may use the code information as a specific evidence. In this case, when the ID is stored in the information code which is read, the medical device information specifying module 31 permits access to the above-described various databases and specifies the medical device information. When the ID is not stored in the information code which is read, the medical device information specifying module 31 denies access to various databases and does not execute specifying the medical device information.

An information transmitting module 21 transmits the acquired medical device information to the information terminal 100 (step S47).

The information transmitting/receiving module 150 receives the medical device information. A display module 172 displays the received medical device information (step S48). The processing in step S48 is the same as the processing in step S28 described above. However, in step S48, the display module 172 does not display the position information of the image capturing location but displays position information of a location where the information code is read.

A calling module 51 calls a phone number included in the medical device information (step S49). The processing in step S49 is the same as the processing in step S29 described above.

On the other hand, in step S45, when determining that one ID has not been specified (NO in step S45), the image analyzing module 40 creates an error notification indicating this fact (step S50). In step S50, the error notification created by the image analyzing module 40 creates, for example, information indicating that a plurality of information codes have been read, information indicating that a plurality of medical device IDs have been specified, information indicating a countermeasure, the information code, information indicating a simple illustration on the countermeasure to be superimposed on the information code, or the like. Further, the contents of the error notification created by the image analyzing module 40 can be appropriately changed. The processing in step S50 is substantially the same as the processing in step S30 described above, except that the captured image is replaced into the information code.

The information transmitting module 21 transmits the created error notification to the information terminal 100 (step S51).

The information transmitting/receiving module 150 receives the notification. The display module 172 displays the error notification on its display unit (step S52). The processing in step S52 is the same as the processing in step S32 described above. However, in step S52, the display module 172 displays the error notification in which the contents of the captured image are replaced into the contents of the information code.

The medical staff wearing the information terminal 100 captures the captured image of the medical device again based on the contents of the error notification. In other words, the information terminal 100 executes the above-described processing of step S40 again.

The above is the code reading process.

In the above-described code reading process, the processing executed by the server 10 or the information terminal 100 may be configured to be collaboratively executed by a the server 10 and the information terminal 100. Alternatively, a part or the whole of the processing executed by the server 10 may be configured to be executed by the information terminal 100, or a part or the whole of the processing executed by the information terminal 100 may be configured to be executed by the server 10.

In the above-described embodiments, the image specifying process and the code reading process are performed independently, but a configuration in which both are executed in combination may be adopted. For example, it may be configured to capture the captured image of the medical device and read the information code. By doing so, the specification of the medical device is executed by the captured image and the information code, so that the specification of the medical device can be more reliably executed.

The means and functions described above are realized by reading and executing a predetermined program by a computer (including a CPU, an information processing device, or various terminals). The program is provided, for example, in a form recorded in a computer-readable recording medium such as a flexible disk, a CD (e.g., CD-ROM or the like), a DVD (DVD-ROM, DVD-RAM, or the like), or the like. In this case, the computer reads the program from the recording medium and transfers the program to an internal storage unit or an external storage unit so as to be stored and executed. Furthermore, the program may be, for example, recorded in a storage device (recording medium) such as a magnetic disk, an optical disk, an optical magnetic disk, or the like in advance and be provided from the recording medium to the computer via a communication line.

While the embodiments of the present invention have been described above, the present invention is not limited to the above-described embodiments. In addition, the effects described in the embodiments of the present invention are merely a list of the most preferable effects produced by the present invention, and the effects of the present invention are limited to those described in the embodiments of the present invention.

### DESCRIPTION OF REFERENCE NUMBERS

1: medical device information providing system, 10: server, 100: information terminal

1. A medical device information providing system for accessing a storage unit that stores medical device information to acquire and display the medical device information, the medical device information providing system comprising:
a medical device specifying unit that specifies an ID of a medical device appearing in a captured image captured by a terminal by image recognition;
a countermeasure acquiring unit that acquire, when a plurality of medical devices appear in the captured image and an ID of one medical device is not specified, a countermeasure for specifying the one medical device;
a countermeasure display unit that displays the acquired countermeasure on a display unit;
a medical device information acquiring unit that acquires, when the ID of the one medical device is specified, medical device information associated with the specified ID of the medical device from the storage unit; and
a display unit that displays the acquired medical device information on the display unit.

2. A medical device information providing system for accessing a storage unit that stores medical device information to acquire and display the medical device information, the medical device information providing system comprising:
a medical device specifying unit that specifies an ID of a medical device associated with a two-dimensional code read by a terminal;
a countermeasure acquiring unit that acquire, when a plurality of two-dimensional codes are read and an ID of one medical device is not specified, a countermeasure for specifying the one medical device;
a countermeasure display unit that displays the acquired countermeasure on a display unit;
a medical device information acquiring unit that acquires, when the ID of the one medical device is specified, medical device information associated with the specified ID of the medical device from the storage unit; and
a display unit that displays the acquired medical device information on a display unit.

3. The medical device information providing system according to claim 1, wherein the display unit displays a change in the acquired medical device information as a graph.

4. The medical device information providing system according to claim 1, wherein the display unit displays the acquired medical device information in association with position information of a location where the terminal captures the captured image.

5. The medical device information providing system according to claim 2, wherein the display unit displays the acquired medical device information in association with position information of a location where the terminal reads the two-dimensional code.

6. The medical device information providing system according to claim 1, further comprising an evidence utilization unit that uses the captured image as an evidence of the specified medical device when accessing the storage unit.

7. The medical device information providing system according to claim 2, further comprising an evidence utilization unit that uses the two-dimensional code as an evidence of the specified medical device when accessing the storage unit.

8. The medical device information providing system according to claim 1, further comprising a calling unit that calls to a phone number included in the acquired medical device information.

9. A medical device information providing method for accessing a storage unit that stores medical device information to acquire and display the medical device information, the medical device information providing method comprising:
specifying an ID of a medical device appearing in a captured image captured by a terminal by image recognition;
acquiring, when a plurality of medical devices appear in the captured image and an ID of one medical device is not specified, a countermeasure for specifying the one medical device;
displaying the acquired countermeasure on a display unit;
acquiring, when the ID of the one medical device is specified, medical device information associated with the specified ID of the medical device from the storage unit; and
displaying the acquired medical device information on the display unit.

10. A medical device information providing method for accessing a storage unit that stores medical device information to acquire and display the medical device information, the medical device information providing method comprising:
specifying an ID of a medical device associated with a two-dimensional code read by a terminal;
acquiring, when a plurality of two-dimensional codes are read and an ID of one medical device is not specified, a countermeasure for specifying the one medical device;
displaying the acquired countermeasure on a display unit;
acquiring, when the ID of the one medical device is specified, medical device information associated with the specified ID of the medical device from the storage unit; and
displaying the acquired medical device information on a display unit.

11. A program for causing a medical device information providing system for accessing a storage unit that stores medical device information to acquire and display the medical device information to execute:
specifying an ID of a medical device appearing in a captured image captured by a terminal by image recognition;
acquiring, when a plurality of medical devices appear in the captured image and an ID of one medical device is not specified, a countermeasure for specifying the one medical device;
displaying the acquired countermeasure on a display unit;
acquiring, when the ID of the one medical device is specified, medical device information associated with the specified ID of the medical device from the storage unit; and
displaying the acquired medical device information on the display unit.

12. A program for causing a medical device information providing system for accessing a storage unit that stores medical device information to acquire and display the medical device information to execute:
specifying an ID of a medical device associated with a two-dimensional code read by a terminal;
acquiring, when a plurality of two-dimensional codes are read and an ID of one medical device is not specified, a countermeasure for specifying the one medical device;
displaying the acquired countermeasure on a display unit;
acquiring, when the ID of the one medical device is specified, medical device information associated with the specified ID of the medical device from the storage unit; and
displaying the acquired medical device information on a display unit.

13. The medical device information providing system according to claim 2, wherein the display unit displays a change in the acquired medical device information as a graph.

14. The medical device information providing system according to claim 2, further comprising a calling unit that calls to a phone number included in the acquired medical device information.
